(19) United States
(12) Patent Application Publication
(10) Pub. No.: US 2021/0386680 A1
(43) Pub. Date: Dec. 16, 2021

(54) SELECTIVELY CLEAVABLE THERAPEUTIC NANOPARTICLES

(71) Applicant: BOARD OF REGENTS, THE UNIVERSITY OF TEXAS SYSTEM, Austin, TX (US)

(72) Inventors: Zhengrong CUI, Austin, TX (US); Abdulaziz ALDAYEL, Austin, TX (US)

(21) Appl. No.: 17/311,159

(22) PCT Filed: Dec. 6, 2019
(86) PCT No.: PCT/US2019/064840

§ 371 Date: Jun. 4, 2021

Related U.S. Application Data
(60) Provisional application No. 62/776,005, filed on Dec. 6, 2018.

(51) Int. Cl. A61K 9/51; A61K 38/38; C07K 16/28; C07K 16/24

(52) U.S. Cl. CPC A61K 9/5146; A61K 38/38; A61K 9/5192; C07K 16/241; C07K 16/2818

(57) ABSTRACT

Disclosed herein are nanoparticles for therapeutic and diagnostic use. The nanoparticles are designed to selectively accumulate in a tissue or organ of interest, and then release one or more therapeutically active or diagnostic agents. The nanoparticles include self-crosslinked therapeutic agents and therapeutic agents dispersed in a matrix.

### CROSS-REFERENCE TO RELATED APPLICATION

This application claims the benefit of U.S. Provisional Application 62/776,005, filed Dec. 6, 2018, the contents of which are hereby incorporated in its entirety.

### STATEMENT OF GOVERNMENT SUPPORT

This invention was made with government support under Grant no. CA135274 awarded by the National Institutes of Health. The government has certain rights in the invention.

### FIELD OF THE INVENTION

The invention is directed to nanoparticles containing one or more therapeutic agents. The nanoparticles selectively accumulate in a specified target tissue, at which point they release the active agent.

### BACKGROUND

Monoclonal antibodies (mAbs) are an important class of therapeutic proteins which are used to treat a wide number of diseases, including cancers, autoimmune disorders, and inflammatory conditions. However, mAb-based medicines also have limitations that impact their clinical use; the most prominent challenges are their unfavorable pharmacokinetic properties and stability issues during manufacturing, transport and storage. Moreover, selective delivery of a mAb to a specific tissue remains an elusive goal. mAbs are typically administered parenterally (intramuscularly, subcutaneously or intravenously) and therefore the majority of the mAb is distributed in the plasma, rather than at the target tissue. Complicating matters, many mAbs suffer from relatively short in vivo half-lives, which necessitate frequent dosing in order to achieve meaningful concentrations of the mAb at the target tissue.

There remains a need for improved formulations for mAbs and other therapeutic agents. There remains a need for improved methods of selectively delivering mAbs and other therapeutic agents to specific tissue sites.

### SUMMARY

Disclosed herein are improved pharmaceutical nanoparticle formulations for selectively delivering mAbs and other therapeutic agents to target tissues. The nanoparticles are selectively accumulated at inflammation sites and tumor tissues, where they are disintegrated thereby releasing the therapeutic agents.

The details of one or more embodiments are set forth in the descriptions below. Other features, objects, and advantages will be apparent from the description and from the claims.

### BRIEF DESCRIPTION OF THE FIGURES

FIG. 1. In vivo longitudinal bioluminescence imaging of acute and chronic inflammation in the right rear foot pad, but not the left rear foot pad, of mice. We induced local tissue inflammation by s.c. injection of 50 μg LPS. (A) Mice were given an i.p. injection of luminol (100 mg/kg) and imaged on day 3. (B) Mice were given an i.p. injection of lucigenin (25 mg/kg) and imaged on day 8.

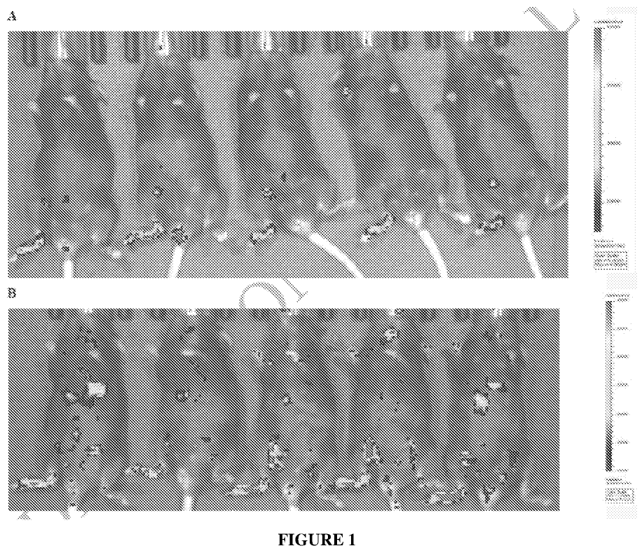

FIG. 2. Physical characterization of the PEGylated gold nanoparticles. (A-B) Representative TEM images of the selected 10 and 100 nm PEGylated gold nanoparticles. (C) Confirmation of PEG on the surface of 2 nm, 10 nm, 20 nm, 50 nm, 80 nm and 100 nm gold nanoparticles. Shown are OD490 values after samples were reacted with Lugol's solution. Data are mean±S.E.M. (n=3).

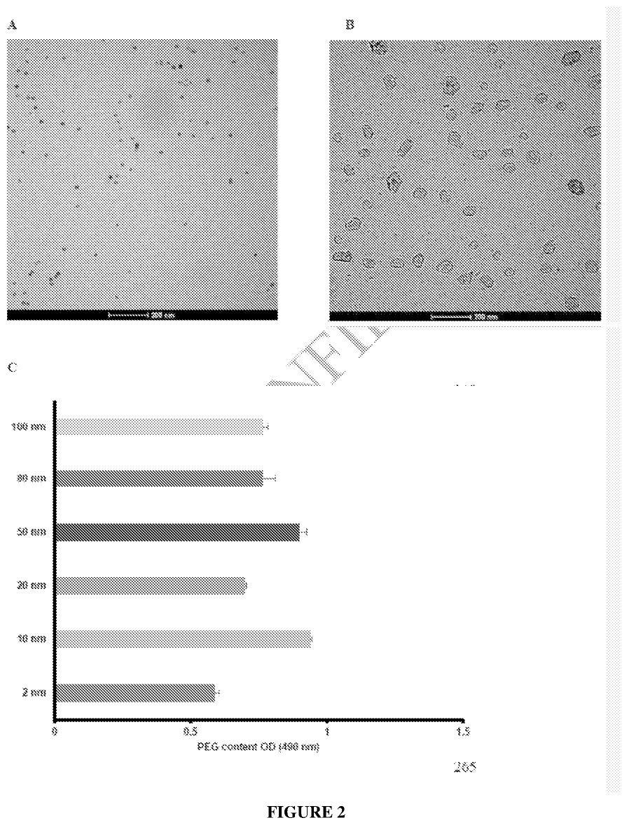

FIG. 3: Pharmacokinetics of 2 nm, 10 nm, 20 nm, 50 nm, 80 nm, 100 nm and 200 nm fluorescently labeled gold nanoparticles in the inflamed foot in mice. Shown are in vivo fluorescence intensity-time profiles and selected pharmacokinetic parameters. Data are mean±S.E. (n=3-5). (a-c, p<0.05).

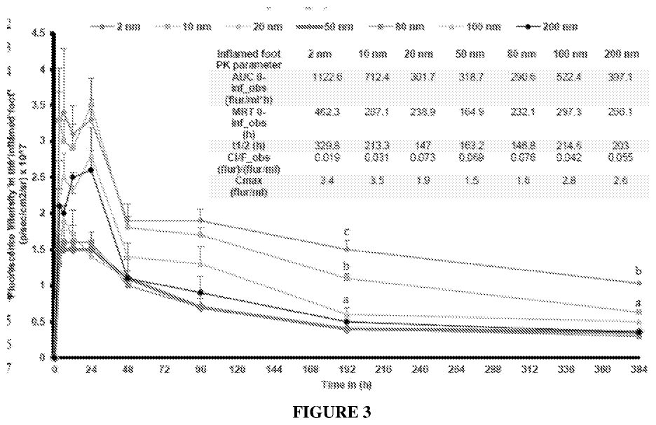

FIG. 4: Specificity towards the inflamed foot relative to the healthy foot and biodistribution of 2 nm, 10 nm, 20 nm, 50 nm, 80 nm, 100 nm and 200 nm fluorescent nanoparticles in other major organs. (A) In vivo fluorescence images of inflamed mouse feet vs. healthy feet at 24 h after i.v. injection of 2 nm, 10 nm, 100 nm and 200 nm nanoparticles. IF=Inflamed Foot. HF=Healthy Foot. (B) Specificity towards the inflamed foot for the 2 nm, 10 nm, 20 nm, 50 nm, 80 nm, 100 nm and 200 nm at 24 h post i.v. injection. The percent of specificity was determined by subtracting the fluorescence intensity value of the healthy foot from the value of the inflamed foot, dividing by the value of the healthy foot, and then multiplying by 100.

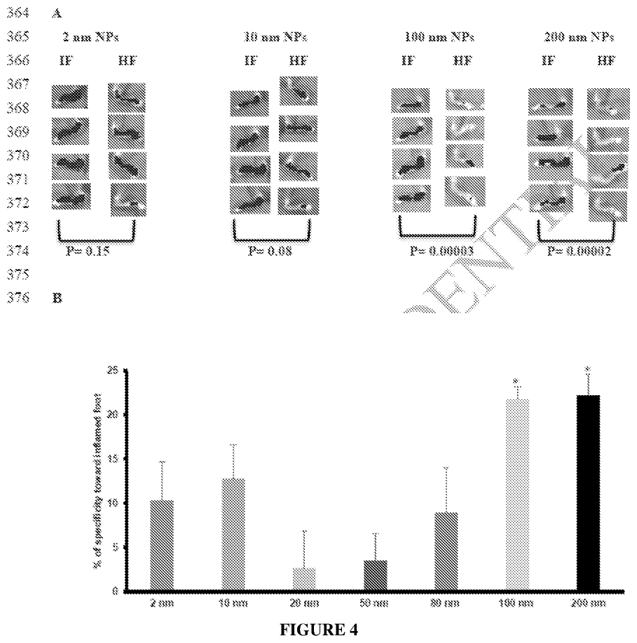

FIG. 5: Normalized fluorescence intensity values in major organs of mice 16 days after i.v. injection of gold nanoparticles. The gold nanoparticles were PEGylated and labeled with Cy 7.5. Data are mean±S.E. (n=4). (*p<0.05) or (a-c, p<0.05).

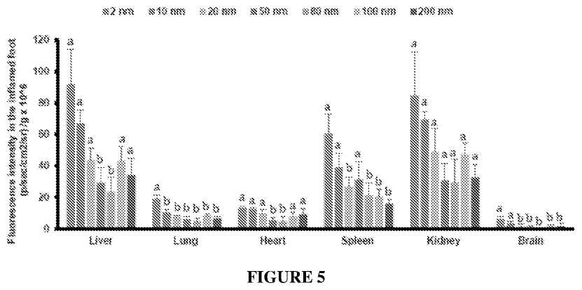

FIG. 6: IgG and IgM specificity towards the inflamed foot relative to the healthy foot within the same mouse with chronic inflammation. (A) IgG fluorescence intensity profile in the rear feet of the mice. (B) IgM fluorescence intensity profile in the rear feet of the mice. (C) Profiles of IgG and IgM fluorescence intensity ratios of inflamed/healthy foot. Data are mean±S.E. (n=4). (*p<0.05).

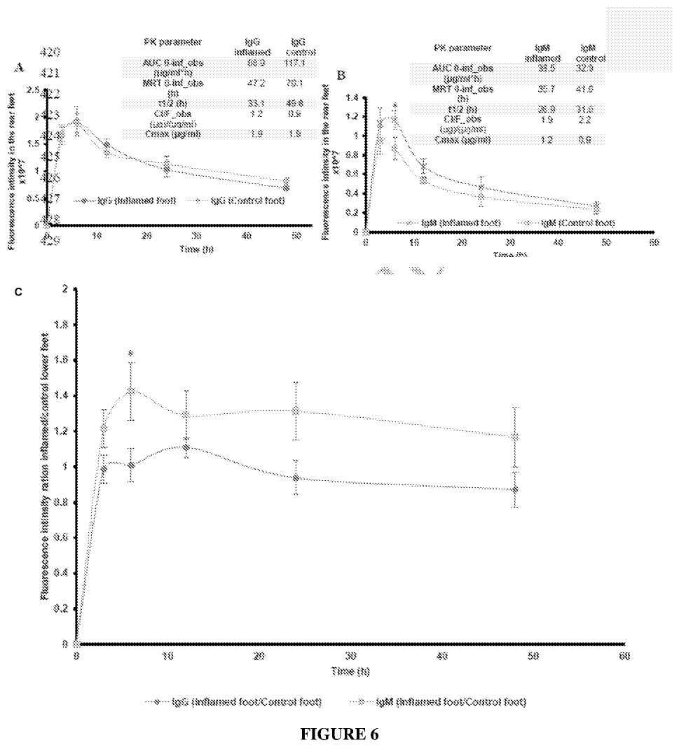

FIG. 7: IgG and IgM specificity towards inflamed foot relative to healthy foot within the same mouse with acute inflammation. (A) IgG fluorescence intensity profile in the rear feet of the mice. (B) IgM fluorescence intensity profile in the rear feet of the mice. (C) Selected pharmacokinetic parameters of IgG and IgM, *Percentage of increase (+) or decrease (−) relatively to the healthy foot. (D) The percent of specificity was determined by subtracting the fluorescence intensity value of healthy foot from the value of the inflamed foot, dividing by the value of the healthy foot, and then multiplying by 100. Data are mean±S.E. (n=6). (*p<0.05).

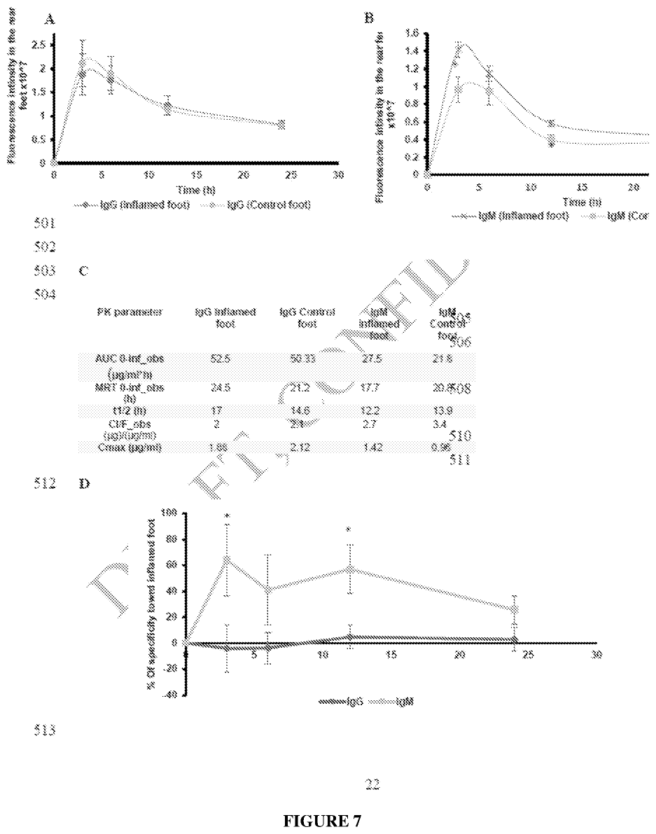

FIG. 8. In vitro characterization and redox-sensitivity of the DTSSP-albumin nanoparticles. (A) TEM image of stable-albumin nanoparticles. (B) TEM image of DTSSP-albumin nanoparticles. (C) In vitro release of albumin from the stable-albumin nanoparticles and the DTSSP-albumin nanoparticles 2 h after pre-incubation in PBS or 1% 2-mercaptoethanol. Data are mean±S.E. (n=3).

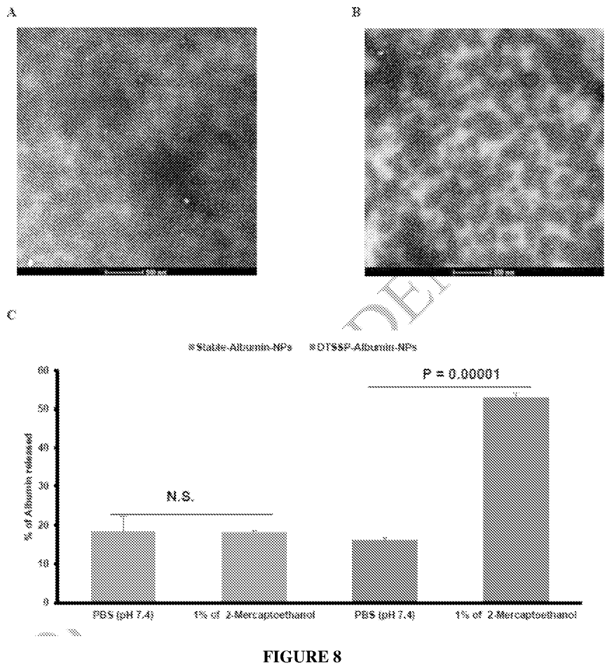

FIG. 9: Specificity and retention of free albumin, stable-albumin nanoparticles and DTSSP-albumin nanoparticles in the inflamed mouse foot. (A) In vivo specificity profile towards the inflamed foot of free albumin, stable-albumin nanoparticles, and DTSSP-albumin nanoparticles within 24 h post i.v. injection. The percent of specificity was determined by subtracting the fluorescence intensity values of the healthy foot from the values of the inflamed foot, then subtracting 1 and multiplying by 100. (B) In vivo fluorescence intensity values measured in the inflamed foot on days 6 and 7 after i.v. injection. (C) A representative in vivo fluorescence images of the inflamed mouse feet on day 6 after i.v. injection. Albumin from bovine serum (BSA) is conjugated to Alexa Fluor™ 680. (D) Uptake and/or binding of fluorescein-labeled albumin by J774A.1 macrophages. J774A.1 cells (2×10⁵) were seeded. Twenty hours later, the medium was replaced with serum-free DMEM containing fluorescein-labeled free albumin or albumin nanoparticles. The cells were washed after 50 min of incubation and lysed, and the fluorescence intensity was measured. Data are mean±S.E. (n=3-5). (A-C, p<0.05) or (*, p<0.05).

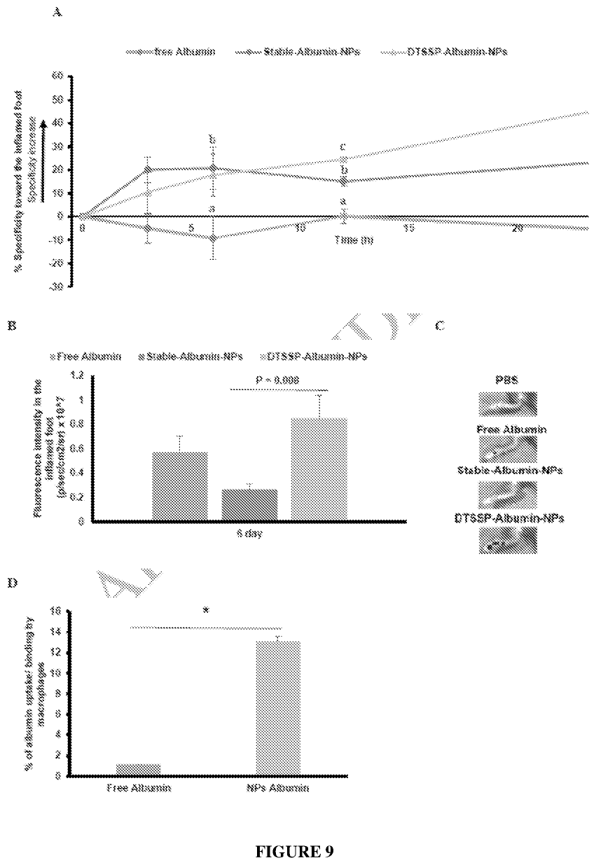

FIG. 10: In vitro characterization of the DTSSP-IgG nanoparticles. (A) TEM image of the free IgG. (B) TEM image of the DTSSP-IgG nanoparticles.

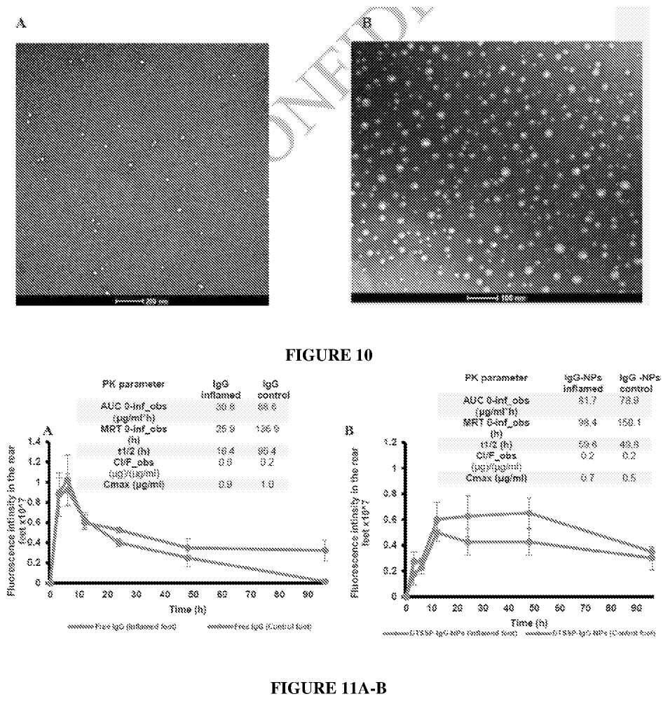

FIG. 11: Selected IgG and DTSSP-IgG-NPs PK parameters and the specificity of them towards the inflamed foot relative to the healthy foot within the same mouse with chronic inflammation. (A) IgG fluorescence intensity profile with selected pharmacokinetic parameters in the rear feet of the mice. (B) DTSSP-IgG-NPs fluorescence intensity profile with selected pharmacokinetic parameters in the rear feet of the mice. (C) Profiles of IgG and DTSSP-IgG-NPs fluorescence intensity ratios of inflamed/healthy foot. Data are mean±S.E. (n=4). (*p<0.05).

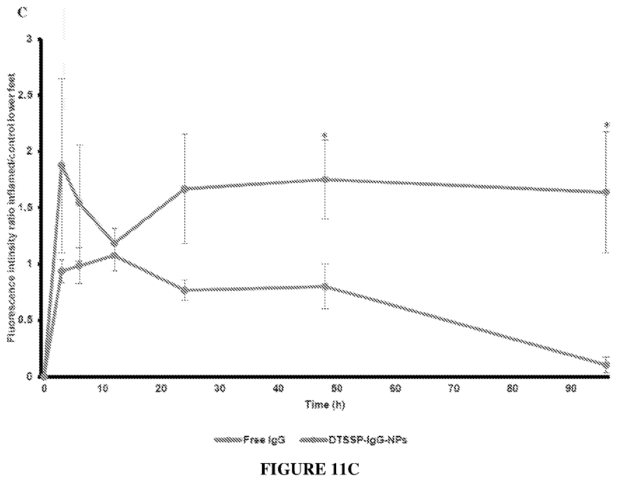

FIG. 12: IgG and IgM distribution in mice with M-Wnt tumors. (A) Percent of IgG or IgM detected in tumors, blood, and other key organs 24 h after i.v. injection in M-Wnt tumor-bearing mice. Shown are percent of dosed fluorescence intensity normalized to the weight of organs and tumors, or the volume of the blood. Values were after subtracting the mean values from the PBS group. (B) Ratios of IgG and IgM in tumor/organs. Data are mean±S.D. (n=3).

FIG. 13. Distribution IgG, free or in DTSSP-IgG-NPs, in mice with M-Wnt tumors.

(A) Percent of IgG detected in tumors, blood, and other key organs in M-Wnt tumor-bearing mice 24 h after i.v. injection with IgG, free or in DTSSP-IgG-NPs. Shown are percent of dosed fluorescence intensity normalized to the weight of organs and tumors, or the volume of the blood. Values were after subtracting the mean values from the PBS group. (B) Ratios of IgG in tumor/organs. Data are mean±S.D. (n=3).

FIG. 14: TNF-α mAb released from DTSSP-TNF-α mAb nanoparticles is still functional and effective in binding to mouse TNF-α. The functionality of the DTSSP-TNF-α mAb nanoparticles is dependent on the concentration of a reducing agent such as GSH, which is known to be higher in synovial fluids and in some tumors, such as breast, ovarian, head and neck and lung cancer, than in blood or other healthy tissues. Data are mean±S.D. (n=3). Data presented for three different mouse TNF-α concentrations; for each concentration leftmost bar: free anti-TNF-α; middle bar: GT003-anti-TNF-α- at high GTH level; rightmost bar: GT003-anti-TNF-α- at low GTH level

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Disclosed herein are therapeutic selectively cleavable nanoparticles that include at least one physiologically active agent. After administration to a patient in need thereof, the nanoparticles accumulate in a specific target tissue, e.g., tumor or inflamed tissues. Once the nanoparticles have reached the target tissue they are selectively disintegrated thereby releasing the active agent at the desired site.

The nanoparticle disclosed herein can have a variety of different particle sizes, depending on the exact target tissue. In some embodiments, the nanoparticles can have an average particle size (d50) from about 50-1,000 nm, from about 100-1,000 nm, from about 100-900 nm, from about 100-800 nm, from about 100-700 nm, from about 100-600 nm, from about 100-500 nm, from about 100-400 nm, from about 100-300 nm, from about 100-200 nm, from about 200-900 nm, from about 200-800 nm, from about 200-700 nm, from about 200-600 nm, from about 200-500 nm, from about 200-400 nm, from about 200-300 nm, from about 300-900 nm, from about 300-800 nm, from about 300-700 nm, from about 300-600 nm, from about 300-500 nm, or from about 300-400 nm.

A variety of different agents can be included in the nanoparticles. In some instances, the agent is a therapeutic agent (e.g., a therapeutic protein, peptide, small molecule, aptamer, or nucleic acid), which in other instances the agent has a diagnostic purpose, for instance a tracer element (e.g., a dye, a radionuclide, contrast agent, and the like). A preferred agent is a therapeutic protein, which includes PEGylated proteins, antibodies, and monoclonal antibodies (“mAbs”). The therapeutic protein can have a variety of different molecular weights. For instance, the therapeutic protein can have a molecular weight between about 10,000 Da and 100,000 kDa, between about 100,000 Da and 100,000 kDa, between about 500,000 Da and 100,000 kDa, between about 1-100,000 kDa, between about 1-75,000 kDa, between about 1-50,000 kDa, between about 1-25,000 kDa, between about 1-10,000 kDa, between about 1-5,000 kDa, between about 1-2,500 kDa, between about 1-1,000 kDa, between about 1-500 kDa, between about 10-500 kDa, between about 20-500 kDa, between about 20-400 kDa, between about 20-300 kDa, between about 20-200 kDa, between about 20-150 kDa, between about 20-100 kDa, between about 20-75 kDa, between about 20-50 kDa, between about 50-500 kDa, between about 50-400 kDa, between about 50-300 kDa, between about 50-200 kDa, between about 50-150 kDa, between about 50-100 kDa, between about 50-75 kDa, between about 100-400 kDa, between about 100-300 kDa, or between about 100-200 kDa.

The nanoparticles disclosed herein can include any number of different monoclonal antibodies. For instance, abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atidortoxumab, atinumab, atorolimumab, avelumab, azintuxizumab vedotin, bapineuzumab, basiliximab, bavituximab, BCD-100, bectumomab, begelomab, belantamab mafodotin, belimumab, bemarituzumab, benralizuma, fasenramab, berlimatoxumab, bermekimab, bersanlimab, bertilimumab, besilesomab, bevacizumab, bezlotoxumab, biciromab, bimagrumab, bimekizumab, birtamimab, bivatuzumab mertansine, bleselumab, blinatumomab, blontuvetmab, blosozumab, bococizumab, brazikumab, brentuximab vedotin, briakinumab, brodalumab, brolucizumab, brontictuzumab, burosumab, crysvitamab, cabiralizumab, camidanlumab tesirine, camrelizumab, canakinumab, cantuzumab mertansine, cantuzumab ravtansine, caplacizumab, capromab pendetide, carlumab, carotuximab, catumaxomab, CBR96-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, CR6261, crenezumab, crizanlizumab, crotedumab, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, diridavumab, domagrozumab, dorlimomab aritox, dostarlimab, drozitumab, ds-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, hemlibra, enapotamab vedotin, enavatuzumab, enfortumab vedotin, enlimomab pegol, enoblituzumab, enokizumab, enoticumab, ensituximab, epitumomab cituxetan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, fbta05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imgatuzumab, inclacumab, indatuximab ravtansine, indusatumab vedotin, inebilizumab, infliximab, inolimomab, inotuzumab ozogamicin, intetumumab, IOMAB-B, ipilimumab, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, lilotomab satetraxetan, lintuzumab, lirilumab, lodelcizumab, lokivetmab, loncastuximab tesirine, lorvotuzumab mertansine, losatuxizumab vedotin, lucatumumab, lulizumab pegol, lumiliximab, lumretuzumab, lupartumab amadotin, lutikizumab, mapatumumab, margetuximab, marstacimab, maslimomab, matuzumab, mavrilimumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab lucentis, ravagalimab, ravulizumab, raxibacumab, refanezumab, regavirumab, relatlimab, remtolumab, reslizumab, rilotumumab, rinucumab, risankizumab, rituximab, rivabazumab pegol, rmabrabishield, robatumumab, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sarilumab, satralizumab, satumomab pendetide, secukinumab, selicrelumab, seribantumab, setoxaximab, setrusumab, sevirumab, SGN-CD19a, SHP647, sibrotuzumab, sifalimumab, siltuximab, simtuzumab, siplizumab sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, talacotuzumab, talizumab, tamtuvetmab, tanezumab, taplitumomab paptox, tarextumab, tavolimab, tefibazumab, telimomab aritox, telisotuzumab vedotin, tenatumomab, teneliximab, teplizumab, tepoditamab, teprotumumab, tesidolumab, tetulomab, tezepelumab, TGN1412, tibulizumab, tigatuzumab, tildrakizumab, timigutuzumab, timolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimabmab, vandortuzumab vedotin, vantictumab, vanucizumab, vapaliximab, varisacumab, varlilumab, vatelizumab, vedolizumab, entyvio, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, zolimomab aritox, and ponezumab can all be advantageously formulated into the inventive nanoparticles.

In some instances, the nanoparticle can include a therapeutic protein, for instance Lepirudin, Dornase alfa, Denileukin diftitox, Bivalirudin, Leuprolide, Peginterferon alfa-2a, Alteplase, Interferon alfa-n1, Darbepoetin alfa, Reteplase, Epoetin alfa, Salmon Calcitonin, Interferon alfa-n3, Pegfilgrastim, Sargramostim, Secretin, Peginterferon alfa-2b, Asparaginase, Thyrotropin Alfa, Antihemophilic Factor, Anakinra, Gramicidin D, Intravenous Immunoglobulin, Anistreplase, Insulin Regular, Tenecteplase, Menotropins, Interferon gamma-1b, Interferon Alfa-2a, Recombinant, Coagulation factor VIIa, Oprelvekin, Palifermin, Glucagon recombinant, Aldesleukin, Botulinum Toxin Type B, Lutropin alfa, Insulin Lispro, Insulin Glargine, Collagenase, Rasburicase, Imiglucerase, Alpha-1-proteinase inhibitor, Pegaspargase, Interferon beta-1a, Pegademase bovine, Human Serum Albumin, Eptifibatide, Serum albumin iodonated, Follitropin beta, Vasopressin, Interferon beta-1b, Hyaluronidase, Insulin, porcine, Digoxin Immune Fab (Ovine), Daptomycin, Pegvisomant, Botulinum Toxin Type A, Pancrelipase, Streptokinase, Alglucerase, Laronidase, Urofollitropin, Serum albumin, Choriogonadotropin alfa, Antithymocyte globulin, Filgrastim, Coagulation factor ix, Becaplermin, Agalsidase beta, Interferon alfa-2b, Oxytocin, Enfuvirtide, Idursulfase, Alglucosidase alfa, Exenatide, Mecasermin, Pramlintide, Galsulfase, Abatacept, Cosyntropin, Corticotropin, Insulin aspart, Insulin detemir, Insulin glulisine, Pegaptanib, Nesiritide, Thymalfasin, Defibrotide, Natural alpha interferon OR multiferon, Glatiramer acetate, Preotact, Teicoplanin, Sulodexide, Teriparatide, Liraglutide, Belatacept, Buserelin, Velaglucerase alfa, Tesamorelin, Taliglucerase alfa, Aflibercept, Asparaginase Erwinia chrysanthemi, Ocriplasmin, Glucarpidase, Teduglutide, Insulin,isophane, Epoetin zeta, Fibrinolysin aka plasmin, Follitropin alpha, Romiplostim, Lucinactant, Aliskiren, Ragweed Pollen Extract, Somatotropin Recombinant, Drotrecogin alfa, Alefacept, OspA lipoprotein, Urokinase, Abarelix, Sermorelin, Aprotinin, Albiglutide, Ancestim, Antithrombin Alfa, Antithrombin III human, Asfotase Alfa, Autologous cultured chondrocytes, Beractant, C1 Esterase Inhibitor (Human), Coagulation Factor XIII A-Subunit (Recombinant), Conestat alfa, Daratumumab, Desirudin, Dulaglutide, Elosulfase alfa, Elotuzumab, Fibrinogen Concentrate (Human), Filgrastim-sndz, Gastric intrinsic factor, Hepatitis B immune globulin, Human calcitonin, Human Clostridium tetani toxoid immune globulin, Human rabies virus immune globulin, Human Rho(D) immune globulin, Hyaluronidase (Human Recombinant), Immune Globulin Human, Turoctocog alfa, Tuberculin Purified Protein Derivative, Simoctocog Alfa, Sebelipase alfa, Sacrosidase, Prothrombin complex concentrate, Poractant alfa, Pembrolizumab, Peginterferon beta-1a, Metreleptin, Methoxy polyethylene glycol-epoetin beta, Insulin Pork, Insulin Degludec, Insulin Beef, Thyroglobulin, Anthrax immune globulin human, Anti-inhibitor coagulant complex, Anti-thymocyte Globulin (Equine), C1 Esterase Inhibitor (Recombinant), Chorionic Gonadotropin (Recombinant), Coagulation factor X human, Efmoroctocog alfa, Factor IX Complex (Human), Hepatitis A Vaccine, Human Varicella-Zoster Immune Globulin, Lenograstim, Pegloticase, Protamine sulfate, Protein S human, Sipuleucel-T, Somatropin recombinant, Susoctocog alfa, Thrombomodulin Alfa, and combinations thereof.

In some embodiments, the active agent itself can be crosslinked into nanoparticle form (i.e., self-crosslinked), while in other embodiments, the active agent can be dispersed in a matrix. The matrix can include crosslinked polymers. In other embodiments, the matrix can include a dispersion of non-covalently bound compounds, either polymers or small molecules. Non-covalent bonds include electrostatic, hydrophobic and van der Waals interactions. Exemplary systems of non-covalently bound nanoparticles include micelles, liposomes, dispersions and conglomerates. Lipids and other self-assembling compounds may be used in non-covalently bound dispersions.

In some embodiments, the crosslinks will include stimuli-cleavable crosslinks. Stimuli-cleavable crosslinks are those which are degraded by exposure to an appropriate trigger, for instance, a catalyst, oxidant, reductant, base, acid, radiation (e.g., UV, infrared, or microwave), ultrasound, heat, or magnetic field. Preferred triggers include oxidants such as reactive oxygen, which are produced in excess in some tumor and inflamed tissues. Exemplary functional groups which can serve as stimuli-cleavable crosslinks include disulfide bonds, trisulfide bonds, diselenide bonds, thioacetals, acetals, oxalates, imines, and short peptide sequences.

Because mAbs and other therapeutic proteins contain a variety of nucleophilic groups, they are especially suitable for self-cross linking into nanoparticles. In some embodiments, the mAb/protein can be dissolved in a suitable solvent and reacted with a crosslinking agent in a stoichiometry suitable to crosslink the mAb/protein into a nanoparticle. The crosslinking agent will contain at least two electrophilic groups capable of reacting with any of the thiol, amine, carboxylate, hydroxyl, or guanidine groups present in the amino acid side chain. In some instances, the crosslinking agent can have the formula:

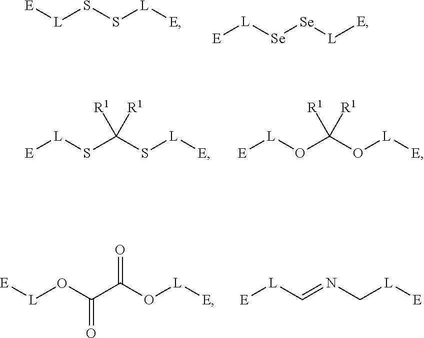

wherein L represent a linking group, R¹ is in each case independently hydrogen or C₁₋₆ alkyl, or where two R¹ groups form a ring; and E is an electrophilic group. Suitable electrophilic groups include imidoester, an N-hydroxysuccinimide ester, a maleimide, a vinyl sulfone, an epoxide, a haloacetyl, or a pyridyl disulfide. In certain embodiments, the crosslinker can include one or more moieties having the formula:

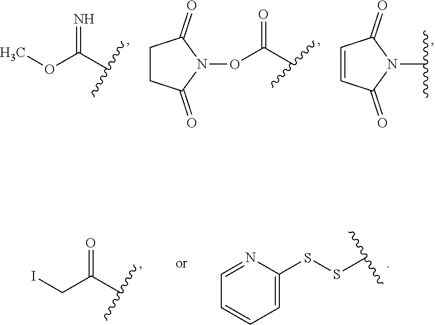

Suitable L groups include C₁₋₁₀alkyl and aryl groups, which may be substituted, or polyethylene glycol chains. The crosslinking agent may be provided in an amount from 10⁻⁵ wt % to 1 wt %, relative to the therapeutic agent. Other suitable ranges include 10⁻⁵ wt % to 0.1 wt %, 10⁻⁵ wt % to 10⁻² wt %; 10⁻⁵ wt % to 10⁻³ wt %; 10⁻⁵ wt % to 10⁻⁴ wt %; 10⁻⁴ wt % to 1 wt %; 10⁻⁴ wt % to 0.1 wt %, 10⁻⁴ wt % to 10⁻² wt %; 10⁻⁴ wt % to 10⁻³ wt %; 10⁻³ wt % to 1 wt %; 10⁻³ wt % to 0.1 wt %, 10⁻³ wt % to 10⁻² wt %; 10⁻² wt % to 1 wt %; and 10⁻² wt % to 0.1 wt %.

In some embodiments, the physiologically active agent is dispersed in a crosslinked polymer matrix, wherein at least a portion of the crosslinks are stimuli-degradable crosslinks, as defined above. Exemplary polymers for crosslinking include polyphosphazenes, polycyanoacrylates, polyesters, polyhydroxyalkanoates, polyanhydrides, polydixanones, polyorthoesters, polyesteramides, polyamido amides, polythioesters, collagen, fibrin, fibrinogen, gelatin, polysaccharides, and combinations thereof. Suitable polyesters include poly(lactic acid), poly(glycolic acid), poly(caprolactone), poly(propylene fumarate), copolymers thereof, and combinations thereof. Suitable polysaccharides include chitosans, celluloses, modified celluloses, alginates, pectins, pullulans, hyaluronic acids, starches, amyloses, and dextrans. These polymers may be crosslinked using the same agents and techniques described about for crosslinking proteins. The crosslinking agent may be provided in an amount from 10⁻⁵ wt % to 1 wt %, relative to polymer to be crosslinked. Other suitable ranges include 1 wt % to 5 wt %, 2.5 wt % to 7.5 wt %, 5 wt % to 10 wt %, 7.5 wt % to 12.5 wt %, 10 wt % to 15 wt %, 12.5 wt % to 17.5 wt %, 15 wt % to 20 wt %, 1 wt % to 30 wt %, 5 wt % to 50 wt %, 10⁻⁵ wt % to 0.1 wt %, 10⁻⁵ wt % to 10⁻² wt %; 10⁻⁵ wt % to 10⁻³ wt %; 10⁻⁵ wt % to 10⁻⁴ wt %; 10⁻⁴ wt % to 1 wt %; 10⁻⁴ wt % to 0.1 wt %, 10⁻⁴ wt % to 10⁻² wt %; 10⁻⁴ wt % to 10⁻³ wt %; 10⁻³ wt % to 1 wt %; 10⁻³ wt % to 0.1 wt %, 10⁻³ wt % to 10⁻² wt %; 10⁻² wt % to 1 wt %; and 10⁻² wt % to 0.1 wt %.

In some embodiments, the nanoparticles will be held together using non-covalent interactions. A preferred system includes acid-sensitive lipids, which can be agglomerated into nanoparticles containing one or more active agents, and which selectively degrade at pH levels lower than found in healthy, non-gastric tissue. Suitable agglomerates include micelles, liposomes, and non-ordered clusters. Exemplary acid sensitive lipids include 1,2-dipalmitoyl-sn-glycero-3-succinate, 1,2-dioleoyl-sn-glycero-3-phosphoethanolamine, 1,2-dioleoyl-sn-glycero-3-succinate, N-palmitoyl homocysteine, cholesteryl hemisuccinate, N-(4-carboxybenzyl)-N,N-dimethyl-2,3-bis(oleoyloxy)propan-1-aminium, PEG-poly(monomethylitaconate)CholC6, and others. The agglomerates can further include a stabilizer, for instance a cholesterol or succinate derivative, e.g., cholesterol hemisuccinate, tocopherol hemisuccinate.

In some embodiments, especially involving small molecule therapeutics, the active agent can be loaded onto smaller nanoparticles, e.g., having a particle size less than 100 nm, less than 75 nm, less than 50 nm, less than 25 nm, less than 10 nm, or less than 5 nm, and then incorporated into the stimuli-cleavable nanoparticles as described above. The larger delivery nanoparticle ensures the agent is preferentially delivered to tumor or inflammation sites, and the smaller nanoparticle increases the persistence of the agent subsequent to the disintegration of the larger delivered nanoparticles.

In certain embodiments, the crosslinks are cleaved by irradiation, for instance x-ray irradiation. A composition can be administered to a patient, either systemically or locally to a desired tissue or tumor location. Once a therapeutic concentration of nanoparticles has accumulated in the tumor or tissue of interest, the tumor or tissue of interest can be exposed to irradiation, for instance, x-ray irradiation, to cleave the nanoparticle and release the active agent. The total amount of irradiation applied can be between 0.1-100 Gy, between 1-50 Gy, between 1-25 Gy, between 1-15 Gy, between 1-10 Gy, between 5-15 Gy, between 10-20 Gy, between 10-30 Gy, between 10-40 Gy, or between 15-50 Gy.

### EXAMPLES

The following examples are for the purpose of illustration of the invention only and are not intended to limit the scope of the present invention in any manner whatsoever.

Lugol's solution, Tris-EDTA (TE), sodium dodecyl sulfate, Triton X-100, N,N-dimethyl-9,9-biacridinium dinitrate (Lucigenin), lipopolysaccharides (LPS) from Salmonella enterica serotype enteritidis, 3-aminophthalhydrazide, 5-amino-2,3-dihydro-1,4-phthalazinedione (Luminol sodium salt), bovine serum albumin (BSA) (lyophilized powder, ≥96%), DTSSP (3,3′-dithiobis(sulfosuccinimidyl propionate)), sulfo-SMCC (sulfosuccinimidyl 4-(N-maleimidomethyl)cyclohexane-1-carboxylate) were from Sigma-Aldrich (St. Louis, Mo.). Albumin-Alexa Fluor™ 647 conjugate and albumin from bovine serum (BSA)-FITC conjugate were from ThermoFisher (Waltham, Mass.). Normal mouse IgG Alexa Fluor® 647 and normal mouse IgM Alexa Fluor® 647 were from Santa Cruz Biotechnology (Dallas, Tex.). Dulbecco's Modified Eagle Medium (DMEM), fetal bovine serum (FBS), and streptomycin/penicillin were from Invitrogen (Carlsbad, Calif.).

### Example 1

PEGylated gold nanoparticles of the following sizes, 2, 10, 20, 50, 80, 100 and 200 nm, labeled with Cy7.5 were from NANOCS (New York, N.Y.). The nanoparticles have a uniform size distribution measured by dynamic light scattering (DLS) and transmission electron microscopy (TEM) by the manufacturer. In addition, the size of the 10 and 100 nm were confirmed using DLS and TEM. Hydrodynamic size and zeta potential were measured using a Malvern ZetaSizer ZS (Westborough, Mass.). For TEM, nanoparticles were deposited onto copper grids, stained with phosphotungstic acid (PTA) (2% w/v) and dried overnight. Before administration, the fluorescence intensity of nanoparticles was measured using IVIS, and nanoparticles that showed higher fluorescent intensity were diluted with PBS so that all the nanoparticles in suspension had a similar fluorescence intensity. The content of polyethylene glycol (2000) (PEG) on the surface of the nanoparticles was measured using an iodide staining method with Lugol's solution. Briefly, 150 μl of nanoparticles (7×10¹² nanoparticles/ml) were added to a solution that contained 950 μl of PBS (pH 7.4, 10 mM) and 68 μl of Lugol's solution. After 5 min of incubation at room temperature, the absorbance (OD490 nm) was measured using a BioTek Synergy HT Multi-Mode Microplate Reader.

BSA was used to formulate the DTSSP-albumin nanoparticles via a desolvation technique. BSA was dissolved at a concentration of 25 mg/ml in 10 mM sodium chloride solution (pH 9.0). The resulting solution was filtered through a 0.22 μm filtration unit (Schleicher and Schüll, Dassel, Germany). An aliquot (1.0 ml) of the BSA solution was transformed into nanoparticles by dropwise addition of 4.0 ml of a desolvating agent (i.e. ethanol/methanol, 50/50%) under stirring (500 rpm) at room temperature. After the desolvation process, 100 μl of 1% DTSSP in water was added to induce protein crosslinking. The crosslinking process was performed over a time period of 24 h at room temperature under stirring. Similarly, stable-albumin nanoparticles were prepared using 100 μl of 1% Sulfo-SMCC as a crosslinker. TEM, nanoparticles were deposited onto copper grids, stained with phosphotungstic acid (PTA) (2% w/v) and dried overnight.

For animal studies, 5 mg of the albumin-Alexa Fluor™ 647 conjugate was added to 20 mg of BSA to prepare fluorescently labeled DTSSP-albumin nanoparticles or stable-albumin nanoparticles. For the uptake study, 1 mg of the albumin-FITC conjugate was added to 24 mg of BSA to prepare fluorescently labeled DTSSP-albumin nanoparticles. The particle size, polydispersity index (PDI), and zeta potential of the nanoparticles were determined using a Malvern Zeta Sizer Nano ZS.

| | Particles size | | Zeta potential |
|---|---|---|---|
| Final formulation | (nm) | PDI | (mV) |
| Stable-Albumin-NPs | 187.3 ± 21 | 0.26 ± 0.02 | −26.0 ± 2.1 |
| DTSSP-Albumin-NPs | 186.3 ± 12 | 0.27 ± 0.03 | −23.7 ± 2.0 |

Data are mean ± SD (n = 3). PDI, polydispersity index.

For in vitro release study, 1% of 2-mercaptoethanol (Hercules, Calif.) was prepared in PBS (10 mM, pH 7.4) to test the stability of the DTSSP-albumin nanoparticles in redox conditions. DTSSP-albumin nanoparticles or stable-albumin nanoparticles were collected by centrifugation (17,500×g, 30 min, 4° C.), resuspended in 1 ml of 1% of 2-mercaptoethanol in PBS or PBS alone (10 mM, pH 7.4), and then placed in shaker incubator (MAQ 5000, MODEL 4350, Thermo Fisher Scientific, Waltham, Mass.) (100 rpm, 37° C.). After 2 h, the tubes were centrifuged (17,500×g, 30 min), and the amount of albumin released (i.e. in the supernatant) was measured using Bradford assay by measuring the absorbance at 595 nm with a BioTek Synergy HT Multi-Mode Microplate Reader.

Normal mouse IgG Alexa Fluor® 647 was used to formulate the DTSSP-IgG-NPs via the desolvation technique as previously described. IgG was diluted at a concentration of 500 μg/ml in 10 mM sodium chloride solution (pH 9.0). The resulting solution was filtered through a 0.22 μm filtration unit. An aliquot (1.0 ml) of the IgG solution was transformed into nanoparticles by dropwise addition of 4.0 ml of a desolvating agent (i.e. ethanol/methanol, 50/50%) under stirring (500 rpm) at room temperature. After the desolvation process, 100 μl of 0.04% DTSSP in water was added to induce particle crosslinking. The crosslinking process was performed under stirring over a time period of 24 h at room temperature. The particle size, polydispersity index (PDI), and zeta potential of the nanoparticles were determined using a Malvern Zeta Sizer Nano ZS. TEM, nanoparticles were deposited onto copper grids, stained with phosphotungstic acid (PTA) (2% w/v) and dried overnight.

Murine macrophage J774A.1 cells (American Type Culture Collection, Manassas, Va.) were seeded in a 12-well plate (2×10⁵ cells/well). To study the effect of the nanoparticle size on their uptake and/or binding by the cells, Free BSA-FITC conjugate or fluorescently labeled DTSSP-albumin nanoparticles were added into the cell culture medium. After 50 min of co-incubation, the cells were washed with PBS (10 mM, pH 7.4) and lysed with a lysis solution that contained 2% (v/v) sodium dodecyl sulfate and 1% Triton X-100. The fluorescence intensity in the cell lysate was measured using a plate reader (Ex=485 nm, Em=528 nm). Bradford protein assay did not show any significant difference in the total protein concentrations in the lysates among the groups.

All animal studies were conducted in accordance with the U.S. National Research Council Guidelines for the care and use of laboratory animals. The animal protocol was approved by the Institutional Animal Care and Use Committee at The University of Texas at Austin. Female C57BL/6 mice (6-8 weeks) were from Charles River Laboratories (Wilmington, Mass.). For imaging, mice were fed with alfalfa-free diet (Harlan, Ind.) to minimize unwanted background signals. An LPS-induced mouse model of chronic inflammation was established follows. Briefly, LPS was dissolved in sterile PBS (pH 7.4, 10 mM) at a concentration of 1 mg/ml. On day 0, 50 μl of the solution was injected into the right hind footpad of each mouse. For the acute inflammation study, acute inflammation was confirmed on day 3 using an IVIS® Spectrum (Caliper, Hopkinton, Mass.) with a bioluminescence imaging system 20 min following intraperitoneal (i.p.) injection of luminol (100 mg/kg) (exposure time 60 s, large binning, field B). For the chronic inflammation studies, chronic inflammation was confirmed on day 8 using an IVIS® Spectrum with a bioluminescence imaging system 20 min following i.p. injection of lucigenin (15 mg/kg) (exposure time 60 s, large binning, field B). Only mice that showed significant acute or chronic inflammation in the right foot were used.

Upon the confirmation of chronic inflammation in the right rear foot, mice were randomly assigned to groups and injected i.v. with PBS or gold nanoparticles of different particle sizes (i.e. 2, 10, 20, 50, 80, 100 and 200 nm). These nanoparticles are non-degradable thus excluding resorption as a variable. Mice were imaged using the IVIS® Spectrum 3, 6, 12, and 24 h and 2, 4, 8, and 16 days after the injection. At the end of the study, mice were euthanized to collect the inflamed foot and major organs (i.e. heart, kidneys, liver, spleen, and lungs). All samples were weighed and imaged using an IVIS® Spectrum. All fluorescent units are in photons per second per centimeter square per steradian (p/s/cm²/sr).

Similarly, groups of mice with chronic inflammation in the right foot were i.v. injected with PBS, free albumin, stable-albumin-NPs or DTSSP-albumin-NPs (albumin-Alexa Fluor™ 647, 0.32 mg/kg). Mice were imaged using an IVIS® Spectrum 3, 6, 12, 24 h and 2, 4, 6 and 7 days after the injection. Data were analyzed using PK Solver. A similar study was also carried out using fluorescently labeled DTSSP-IgG-nanoparticles (IgG, 2 μg/kg). Mice were imaged using IVIS® Spectrum 3, 6, 12, 24 h and 2 and 4 days after the injection.

Finally, the distribution of IgG and IgM, both fluorescently labeled, in acute or chronic inflammation sites after they were i.v. injected in mice with LPS-induced inflammation were studied similarly. Upon confirmation of acute or chronic inflammation in the right rear foot, mice were randomly assigned to groups and i.v. injected with PBS, IgG or IgM (IgM, 40 μg/kg; IgG, 20 μg/kg to account for difference in fluorescence intensities). Mice were imaged using the IVIS® Spectrum 3, 6, 12, and 24 h after the injection to determine specificity of IgG and IgM to the inflammation sites within the first 24 h.

Statistical analyses were completed by performing analysis of variance followed by Fisher's protected least significant difference procedure. A p value of ≤0.05 (two-tail) was considered significant.

### Example 2

Redox-sensitive IgG nanoparticles were prepared as in Example 1. Briefly, normal mouse IgG Alexa Fluor® 647 from Santa Cruz Biotechnology (Dallas, Tex.) was diluted to a concentration of 100 μg/ml in a 10 mM sodium chloride solution, pH 9.0. Aliquots (1.0 ml) of the IgG solution were transformed into nanoparticles by dropwise addition of 4.0 ml of a desolvating agent (i.e. ethanol/methanol, 50%/50%) under stirring (500 rpm) at room temperature. After the desolvation process, 100 μl of a 3,3′-dithiobis(sulfosuccinimidyl propionate) in water solution (i.e. DTSSP, 0.004%) were added to induce particle crosslinking (i.e. 24 h at room temperature under stirring). Particle size was measured using a Malvern Nano ZS and morphology examined using transmission electron microscopy.

M-Wnt mammary tumor cells (basal-like, triple-negative, claudin-low) were cloned from spontaneous mammary tumors in MMTV-Wnt-1 transgenic mice in a congenic C57BL/6 background. M-Wnt cells were cultured in RPMI 1640 medium at 37° C. and 5% CO₂. The medium was supplemented with 10% fetal bovine serum (FBS), 100 U/mL of penicillin, and 100 μg/mL of streptomycin. All cell culture medium and reagents were from Invitrogen (Carlsbad, Calif.). Animal study was conducted in accordance with the U.S. National Research Council Guidelines for the care and use of laboratory animals. The animal protocol was approved by the Institutional Animal Care and Use Committee at The University of Texas at Austin. Female C57BL/6 mice (6-8 weeks) were from Charles River Laboratories (Wilmington, Mass.). M-Wnt tumors were established by injecting M-Wnt tumor cells (5×10⁵ cells/mouse) subcutaneously in the ninth mammary fat pad of the mice. When tumors reached 6-9 mm in diameter, mice were i.v. injected with PBS, IgG, IgM, or DTSSP-IgG. Both IgG and IgM (Sant Cruz Biotechnology) were fluorescently labeled with Alexa Fluor® 647. The dose of IgM was 40 mg/kg, 20 mg/kg for IgG so that the fluorescence intensities of the two antibodies injected in each mouse were similar. Mice were euthanized 24 h later to collect blood, tumor, and major organs (e.g. heart, kidneys, liver, spleen, and lung, gastrointestinal tract). All samples were then imaged using an IVIS Spectrum (Caliper, Hopkinton, Mass.) (Em/Ex of 465/600 nm).

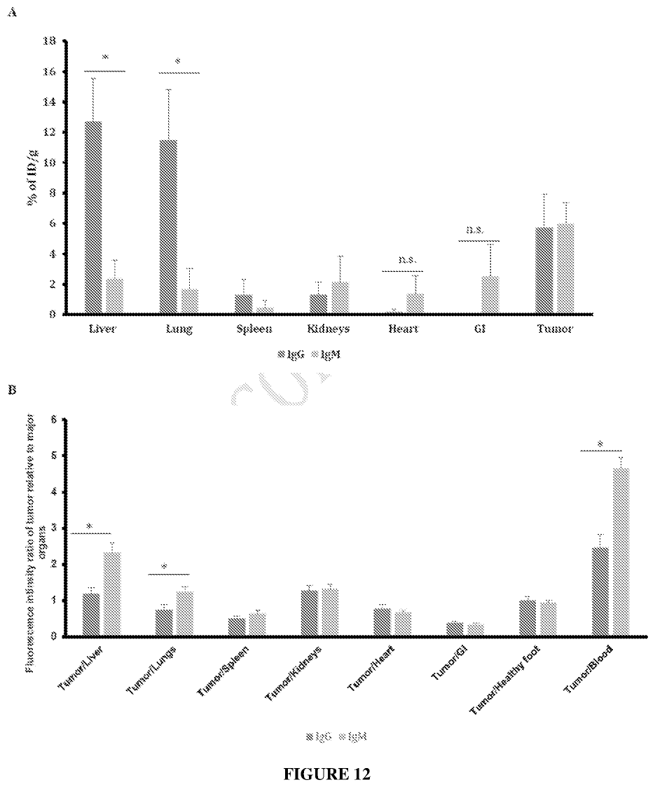

IgG and IgM are natural, large biologic molecules with particle size in the nanometer scale (i.e. IgG, ˜10 nm; IgM, ˜150 nm). To preliminarily study the distribution of IgG and IgM in tumor tissues, relative to other key organs, we injected (i.v.) M-Wnt tumor-bearing mice with fluorescently labeled IgG or IgM. Shown in FIG. 12A are the percentages of injected IgG and IgM that were detected in tumors, blood, and key organs, 24 h after the injection. IgM and IgG showed similar weight-normalized levels in tumor tissues, but the weight- or volume-normalized levels of IgM in the liver, lung, and blood are significantly lower than those of the IgG (FIG. 12A). Shown in FIG. 12B are the ratios of IgG and IgM levels in tumor issues, relative to in liver, lung, and blood, clearly indicating that the IgM has more specific distribution to tumors than IgG.

IgGs, such as anti-PD-1 monoclonal antibodies, are used extensively in clinics to treat various types of cancers, but are associated with severe adverse events, likely relative to their non-specific distribution upon injection. Currently, there is effort in developing anti-PD-1 IgM antibodies. However, as mentioned above, the affinity of IgMs is not as high as IgGs. We therefore tested whether crosslinking IgG into redox-sensitive nanoparticles will increase its specific distribution in tumors. Tumor cells are known to be in a state of redox imbalance, resulting in increased oxidants within the tumor microenvironment. We synthesized redox-sensitive IgG nanoparticles (i.e. DTSSP-IgG-NPs) with a hydrodynamic protein size of about 170±21 nm and i.v. injected them, or free IgG, into mice with M-Wnt tumors.

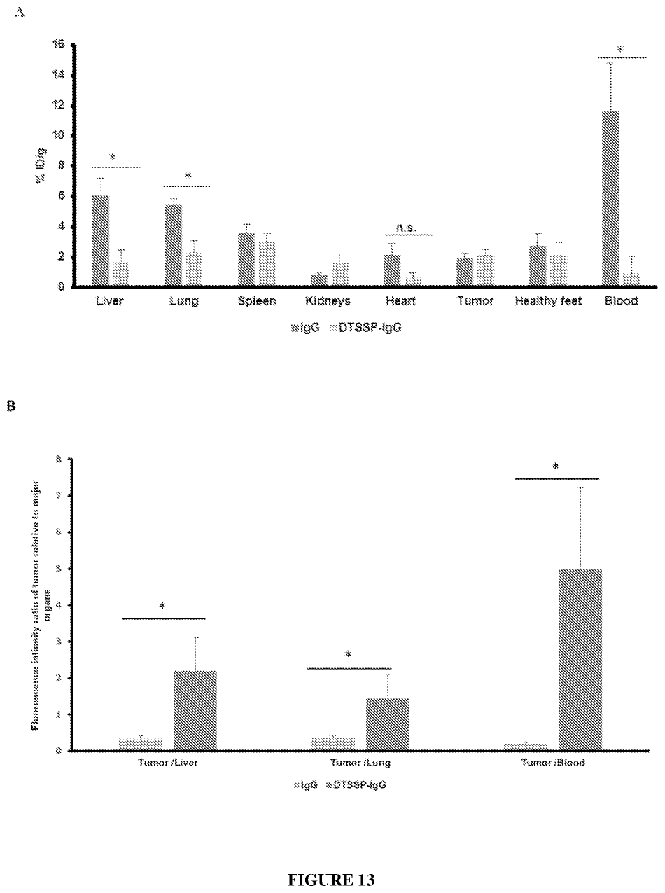

As shown in FIG. 13A, DTSSP-IgG-NPs and IgG have similar levels of weight-normalized distributions in tumors, but the levels of IgG in liver, lung, and blood, when given as DTSSP-IgG-NPs, are significantly lower, compared to when given as free IgG. Shown in FIG. 13B are the ratios of IgG in tumor issues, relative to liver, lung, and blood, 24 h after mice were injected with free IgG or IgG in DTSSP-IgG-NPs, indicating that formulating IgG into DTSSP-IgG-NPs may increase its specific distribution to tumors. Currently, we are testing the affinity of IgG released from the DTSSP-IgG-NPs.

Pulmonary and liver adverse events are commonly associated with monoclonal antibodies that have been approved for clinical use, although the mechanisms underlying such adverse effects are generally not known. For pulmonary adverse effects there are four main categories: interstitial pneumonitis and fibrosis; acute respiratory distress syndrome (ARDS), bronchiolitis obliterans organizing pneumonia (BOOP), and hypersensitivity reactions. Liver is an Fc-receptor rich organ that helps to increase the circulation and retention time of monoclonal antibodies. A possible side effect of antibody therapy is the cytokine-release syndrome that may lead to autoimmune complications via interactions with Fc receptors. Due to the longer exposure, life-threatening and fatal cytokine release syndrome has been reported with antibody therapies (e.g. Rituximab for treatment of chronic lymphocytic leukemia (CLL) and non-Hodgkin's lymphomas). The high concentrations of IgG in lung and liver are probably related to the high residual plasma concentrations of the IgG in those organs, which may also be related to the adverse events caused by monoclonal antibodies in the lung and liver.

### Example 3: Confirmation of the Functionality of TNF-α Released from Redox-Sensitive TNF-α mAb Nanoparticles (DTSSP-TNF-α mAb-Nanoparticles)

Redox-sensitive TNF-α mAb nanoparticles were prepared as in Example 1 and 2. Briefly InVivoMAb anti-mouse TNFα (TNF-α mAb) from Bio-X-Cell (West Lebanon, N.H.) was diluted to a concentration of 1 mg/ml in a 10 mM sodium chloride solution, pH 9.0. Aliquots (1.0 ml) of the TNF-α mAb solution were transformed into nanoparticles by dropwise addition of 4.0 ml of a desolvating agent (i.e. ethanol/methanol, 50%/50%) under stirring (500 rpm) at room temperature. After the desolvation process, 100 μl of a 3,3′-dithiobis(sulfosuccinimidyl propionate) in water solution (i.e. DTSSP, 0.04%) were added to induce particle crosslinking (i.e. 24 h at room temperature under stirring).

The particles were centrifuged at 15,000 rpm for 30 min, then the pellet was suspended in 1 ml solution that contain about 1.6 or 0.8 nmoles of glutathione (Sigma, St. Louis, Mo.). The mixture was then placed in shaker incubator (MAQ 5000, MODEL 4350, Thermo Fisher Scientific, Waltham, Mass.) for about 1.5 h (150 rpm, 37° C.) to allow the TNF-α mAbs to release from the nanoparticles. To determine the ability of TNF-α mAb in binding mouse TNF-α, about 12.5 μg (0.5 ml of 25 μg/ml added to 0.5 ml of the samples) of free TNF-α mAb or redox-sensitive TNF-α mAb nanoparticles were incubated with different concentrations of mouse TNF-α (125, 62.5 and 31.25 μg/ml, final concentration) and then placed in shaker incubator for about 2 h (150 rpm, 37° C.). The concentrations of TNF-α in the samples were measured using a Mouse TNF-α ELISA MAXIM Standard from BioLegend (San Diego, Calif.). Results were expressed as the percent of TNF-α bound by the anti-TNF-α mAb using the following equation:

% TNF-α bound=100×1−(OD of mouse TNF-α bound to anti-TNF-α mAb)/(OD of mouse TNF-α alone).

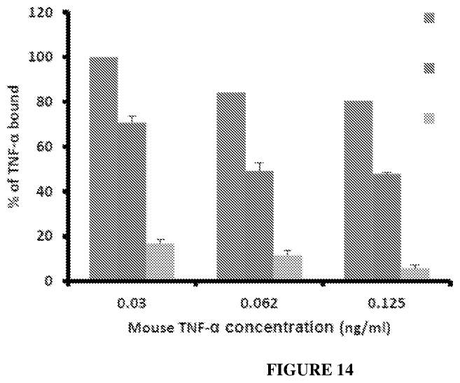

Results are shown in FIG. 14.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A therapeutic nanoparticle comprising stimuli-cleavable crosslinks, and at least one physiologically active agent, wherein the nanoparticle has a particle size of between about 100-1,000 nm, and wherein the nanoparticle undergoes stimulus-directed degradation.

2. The nanoparticle of claim 1, wherein the nanoparticle has a particle size from about 100-400 nm.

3-6. (canceled)

7. The nanoparticle of claim 1, wherein the at least one physiologically active agent comprises at least one antibody or therapeutic protein.

8. The nanoparticle of claim 1, wherein the at least one physiologically active agent comprises at least one monoclonal antibody.

9. The nanoparticle of claim 1, wherein the physiologically active agent is crosslinked.

10. (canceled)

11. The nanoparticle of claim 1, wherein the physiologically active agent is dispersed in a matrix comprising one or more polymers or lipids.

12. The nanoparticle of claim 1, wherein the physiologically active agent is dispersed in a crosslinked polymer matrix, wherein the crosslinked polymer is selected from the group consisting of polyphosphazenes, polycyanoacrylates, polyesters, polyhydroxyalkanoates, polyanhydrides, polydixanones, polyorthoesters, polyesteramides, polyamido amides, polythioesters, collagen, fibrin, fibrinogen, gelatin, polysaccharides, and combinations thereof.

13. The nanoparticle of claim 1, wherein the crosslinked polymer matrix comprises a polyester selected from the group consisting of poly(lactic acid), poly(glycolic acid), poly(caprolactone), poly(propylene fumarate), copolymers thereof, and combinations thereof.

14. The nanoparticle of claim 1, wherein the crosslinked polymer comprises a polysaccharide selected from the group consisting of chitosans, celluloses, modified celluloses, alginates, pectins, pullulans, hyaluronic acids, starches, amyloses, dextrans

15. The nanoparticle of claim 1, wherein the physiologically active agent is loaded into smaller nanoparticles having a particle size no greater than 50 nm, said smaller nanoparticles dispersed in a matrix.

16. The nanoparticle of claim 1, wherein the stimuli-cleavable crosslinks are degraded upon exposure to oxidant, reductant, acid, irradiation, ultrasound, heat or magnetic exposure.

17-22. (canceled)

23. The nanoparticle of claim 1, wherein the stimuli-cleavable crosslinks comprise functional groups selected from disulfide bonds, trisulfide bonds, diselenide bonds, thioacetals, acetals, oxalates, imines, and peptide bonds.

24. The nanoparticle of claim 1, wherein the stimuli-cleavable crosslinks comprise disulfide bonds.

25-38. (canceled)

39. The nanoparticle of claim 1, wherein the at least one physiologically active agent comprises a monoclonal antibody selected from abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atidortoxumab, atinumab, atorolimumab, avelumab, azintuxizumab vedotin, bapineuzumab, basiliximab, bavituximab, BCD-100, bectumomab, begelomab, belantamab mafodotin, belimumab, bemarituzumab, benralizuma, fasenramab, berlimatoxumab, bermekimab, bersanlimab, bertilimumab, besilesomab, bevacizumab, bezlotoxumab, biciromab, bimagrumab, bimekizumab, birtamimab, bivatuzumab mertansine, bleselumab, blinatumomab, blontuvetmab, blosozumab, bococizumab, brazikumab, brentuximab vedotin, briakinumab, brodalumab, brolucizumab, brontictuzumab, burosumab, crysvitamab, cabiralizumab, camidanlumab tesirine, camrelizumab, canakinumab, cantuzumab mertansine, cantuzumab ravtansine, caplacizumab, capromab pendetide, carlumab, carotuximab, catumaxomab, CBR96-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, CR6261, crenezumab, crizanlizumab, crotedumab, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, diridavumab, domagrozumab, dorlimomab aritox, dostarlimab, drozitumab, ds-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, hemlibra, enapotamab vedotin, enavatuzumab, enfortumab vedotin, enlimomab pegol, enoblituzumab, enokizumab, enoticumab, ensituximab, epitumomab cituxetan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, fbta05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imgatuzumab, inclacumab, indatuximab ravtansine, indusatumab vedotin, inebilizumab, infliximab, inolimomab, inotuzumab ozogamicin, intetumumab, IOMAB-B, ipilimumab, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, lilotomab satetraxetan, lintuzumab, lirilumab, lodelcizumab, lokivetmab, loncastuximab tesirine, lorvotuzumab mertansine, losatuxizumab vedotin, lucatumumab, lulizumab pegol, lumiliximab, lumretuzumab, lupartumab amadotin, lutikizumab, mapatumumab, margetuximab, marstacimab, maslimomab, matuzumab, mavrilimumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab lucentis, ravagalimab, ravulizumab, raxibacumab, refanezumab, regavirumab, relatlimab, remtolumab, reslizumab, rilotumumab, rinucumab, risankizumab, rituximab, rivabazumab pegol, rmabrabishield, robatumumab, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sarilumab, satralizumab, satumomab pendetide, secukinumab, selicrelumab, seribantumab, setoxaximab, setrusumab, sevirumab, SGN-CD19a, SHP647, sibrotuzumab, sifalimumab, siltuximab, simtuzumab, siplizumab sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, talacotuzumab, talizumab, tamtuvetmab, tanezumab, taplitumomab paptox, tarextumab, tavolimab, tefibazumab, telimomab aritox, telisotuzumab vedotin, tenatumomab, teneliximab, teplizumab, tepoditamab, teprotumumab, tesidolumab, tetulomab, tezepelumab, TGN1412, tibulizumab, tigatuzumab, tildrakizumab, timigutuzumab, timolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimabmab, vandortuzumab vedotin, vantictumab, vanucizumab, vapaliximab, varisacumab, varlilumab, vatelizumab, vedolizumab, entyvio, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, zolimomab aritox, ponezumab, and combinations thereof.

40. The nanoparticle of claim 1, wherein the physiologically active agent is a therapeutic protein selected from Lepirudin, Dornase alfa, Denileukin diftitox, Bivalirudin, Leuprolide, Peginterferon alfa-2a, Alteplase, Interferon alfa-n1, Darbepoetin alfa, Reteplase, Epoetin alfa, Salmon Calcitonin, Interferon alfa-n3, Pegfilgrastim, Sargramostim, Secretin, Peginterferon alfa-2b, Asparaginase, Thyrotropin Alfa, Antihemophilic Factor, Anakinra, Gramicidin D, Intravenous Immunoglobulin, Anistreplase, Insulin Regular, Tenecteplase, Menotropins, Interferon gamma-1b, Interferon Alfa-2a, Recombinant, Coagulation factor VIIa, Oprelvekin, Palifermin, Glucagon recombinant, Aldesleukin, Botulinum Toxin Type B, Lutropin alfa, Insulin Lispro, Insulin Glargine, Collagenase, Rasburicase, Imiglucerase, Alpha-1-proteinase inhibitor, Pegaspargase, Interferon beta-1a, Pegademase bovine, Human Serum Albumin, Eptifibatide, Serum albumin iodonated, Follitropin beta, Vasopressin, Interferon beta-1b, Hyaluronidase, Insulin, porcine, Digoxin Immune Fab (Ovine), Daptomycin, Pegvisomant, Botulinum Toxin Type A, Pancrelipase, Streptokinase, Alglucerase, Laronidase, Urofollitropin, Serum albumin, Choriogonadotropin alfa, Antithymocyte globulin, Filgrastim, Coagulation factor ix, Becaplermin, Agalsidase beta, Interferon alfa-2b, Oxytocin, Enfuvirtide, Idursulfase, Alglucosidase alfa, Exenatide, Mecasermin, Pramlintide, Galsulfase, Abatacept, Cosyntropin, Corticotropin, Insulin aspart, Insulin detemir, Insulin glulisine, Pegaptanib, Nesiritide, Thymalfasin, Defibrotide, Natural alpha interferon OR multiferon, Glatiramer acetate, Preotact, Teicoplanin, Sulodexide, Teriparatide, Liraglutide, Belatacept, Buserelin, Velaglucerase alfa, Tesamorelin, Taliglucerase alfa, Aflibercept, Asparaginase Erwinia chrysanthemi, Ocriplasmin, Glucarpidase, Teduglutide, Insulin, isophane, Epoetin zeta, Fibrinolysin aka plasmin, Follitropin alpha, Romiplostim, Lucinactant, Aliskiren, Ragweed Pollen Extract, Somatotropin Recombinant, Drotrecogin alfa, Alefacept, OspA lipoprotein, Urokinase, Abarelix, Sermorelin, Aprotinin, Albiglutide, Ancestim, Antithrombin Alfa, Antithrombin III human, Asfotase Alfa, Autologous cultured chondrocytes, Beractant, C1 Esterase Inhibitor (Human), Coagulation Factor XIII A-Subunit (Recombinant), Conestat alfa, Daratumumab, Desirudin, Dulaglutide, Elosulfase alfa, Elotuzumab, Fibrinogen Concentrate (Human), Filgrastim-sndz, Gastric intrinsic factor, Hepatitis B immune globulin, Human calcitonin, Human Clostridium tetani toxoid immune globulin, Human rabies virus immune globulin, Human Rho(D) immune globulin, Hyaluronidase (Human Recombinant), Immune Globulin Human, Turoctocog alfa, Tuberculin Purified Protein Derivative, Simoctocog Alfa, Sebelipase alfa, Sacrosidase, Prothrombin complex concentrate, Poractant alfa, Pembrolizumab, Peginterferon beta-1a, Metreleptin, Methoxy polyethylene glycol-epoetin beta, Insulin Pork, Insulin Degludec, Insulin Beef, Thyroglobulin, Anthrax immune globulin human, Anti-inhibitor coagulant complex, Anti-thymocyte Globulin (Equine), C1 Esterase Inhibitor (Recombinant), Chorionic Gonadotropin (Recombinant), Coagulation factor X human, Efmoroctocog alfa, Factor IX Complex (Human), Hepatitis A Vaccine, Human Varicella-Zoster Immune Globulin, Lenograstim, Pegloticase, Protamine sulfate, Protein S human, Sipuleucel-T, Somatropin recombinant, Susoctocog alfa, Thrombomodulin Alfa, and combinations thereof.

41-51. (canceled)

52. A method of preparing the nanoparticle of claim 9, comprising contacting the physiologically active agent with at least one crosslinking agent at a concentration and time sufficient to prepare a crosslinked nanoparticle.

53. The method of claim 52, wherein the crosslinking agent has the formula: 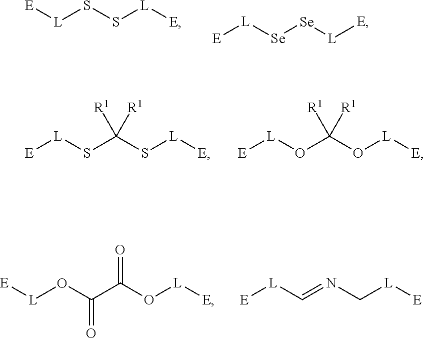
wherein L represent a linking group, R¹ is in each case independently hydrogen or C₁₋₆ alkyl, or where two R¹ groups form a ring; and
E is an electrophilic group.

54. The method of claim 53, wherein E comprises an imidoester, an N-hydroxysuccinimide ester, a maleimide, a haloacetyl, or a pyridyl disulfide.

55. The method of claim 53, wherein E has the formula:

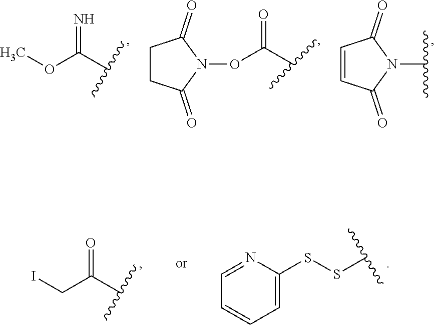

56. (canceled)

57. The method of claim 52, wherein the crosslinking agent is provided in an amount from 0.00001-0.1 wt %, relative to the physiologically active agent.
